(19) United States
(12) Patent Application Publication
(10) Pub. No.: US 2025/0387241 A1
(43) Pub. Date: Dec. 25, 2025

(54) CABLE-CONTROLLED PARTIAL HAND PROSTHESIS

(71) Applicant: PINDO LABS INC, Loveland, CO (US)

(72) Inventor: Nadia SULLIVAN, Loveland, CO (US)

(21) Appl. No.: 18/751,507

(22) Filed: Jun. 24, 2024

(51) Int. Cl. A61F 2/58; A61F 2/68; A61F 2/50

(52) U.S. Cl. CPC A61F 2/586; A61F 2/68; A61F 2002/5072

(57) ABSTRACT

A partial hand prosthesis has a palm socket, a lever, a linkage system, and a spring-cable system. The palm socket is shaped and configured to engage the contours of a user’s hand, the palm socket having a palmar portion configured to engage a palmar portion of the user’s hand and a dorsal portion configured to engage a dorsal portion of the user’s hand. The lever is pivotally coupled to the palm socket, the lever configured to rotate from a first position to a second position in response to a palmar-facing pressure. The linkage system is shaped and configured to simulate a finger-like motion in response to activation of the spring-cable system. The spring-cable system is configured to respond to rotation of the lever.

### TECHNICAL FIELD

Embodiments herein are related to partial hand prostheses.

### BACKGROUND

The prosthetics industry has been active for hundreds of years, pursuing various attempts to provide amputees or those born with missing limbs or digits with improved use of their limbs/digits, and/or aesthetic appeal. In the 19^{th}- and early 20th-centuries, great focus was given to the aesthetic appearance of prostheses, with attempts to provide limbs or digits that appeared real. More recently, in particular with regard to upper limbs/hands, the industry has attempted to provide functioning digits or limbs. Some companies offer specialized devices that are only to be used for one purpose. These devices, however, are so specialized that users typically must resort to carrying multiple devices on their persons, so they could adapt to the day’s activities.

In terms of moving functioning digits, the industry has developed robotic or hydraulic features. These devices have, however, multiple disadvantages. For example, electronic robotic devices require constant access to a power source, while hydraulic devices are typically slow to activate. Moreover, they typically require the user to activate a motion using a hand, such as an opposing hand in the case of a hand prosthesis.

There thus remains a need for a partial hand prosthesis having new and improved features.

### SUMMARY

An exemplary partial hand prosthesis has a palm socket, a lever, a linkage system, and a spring-cable system. The palm socket is shaped and configured to engage the contours of a user’s hand, the palm socket having a palmar portion configured to engage a palmar portion of the user’s hand and a dorsal portion configured to engage a dorsal portion of the user’s hand. The lever is pivotally coupled to the palm socket and configured to rotate from a first position to a second position in response to a palmar-facing pressure. The linkage system is shaped and configured to simulate a finger-like motion in response to activation of the spring-cable system. The spring-cable system is configured to respond to rotation of the lever.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the Background.

### BRIEF DESCRIPTION OF THE DRAWINGS

FIG. 1 is a perspective right-side view of an exemplary partial hand prosthetic;

FIG. 2 is a perspective left-side view thereof;

FIG. 3 is a right-side view of a linkage system for use therewith;

FIG. 4 is a left-side view of the linkage system; and

FIG. 5 is a top view of a portion of an exemplary linkage system suitable for use in the partial hand prosthetic.

### DETAILED DESCRIPTION

Before providing a detailed description of the invention, it is prudent to provide definitions for various terms used herein. For the purpose of this document, the terms “proximal”, “distal”, “dorsal”, and “palmar” shall be understood in the common anatomical sense. That is, for example, the dorsal portion of the palm socket shall be understood to reference the portion of the palm socket that is intended for positioning adjacent to, or associated with, the dorsal portion of the user’s hand. When reference is made to an anatomical feature of a user’s hand, such as, for example, a particular digit, it should be understood that the nearest anatomical area is intended if the user does not have the referenced feature. In the case of a missing digit, the nearest anatomical feature may be an amputation site or a near portion of a hand or palm.

In embodiments described herein, a prosthesis 100 is provided which offers users a fast-acting mechanical means to cause a prosthetic digit to close about an object in response to a lever activation on the same hand, and to automatically release the object when the lever is no longer activated. This mechanical activation provides an intuitive grasping and release action for the user that is not dependent on hydraulics or activation by the opposing hand and improves daily activities for the user not available in currently available products. The prosthesis 100 provides an intuitive mechanical feedback sensation to the wearer: pressure on the wearer’s palm causes the “finger” to close, and releasing pressure causes the “finger” to open; moreover, the greater the pressure, the tighter the grip.

It is explicitly noted that the inventor of embodiments herein, a competitive cyclist, desired to provide a device that can be used to provide a feedback sensation, strength, and stability while riding a bicycle, particularly at high speeds, which require quick activation and release. Currently-available devices specifically adapted for cycling do not offer a mechanism to automatically grasp a handlebar and, subsequently, quickly and automatically release a grip on a handlebar. Embodiments described herein offer this functionality.

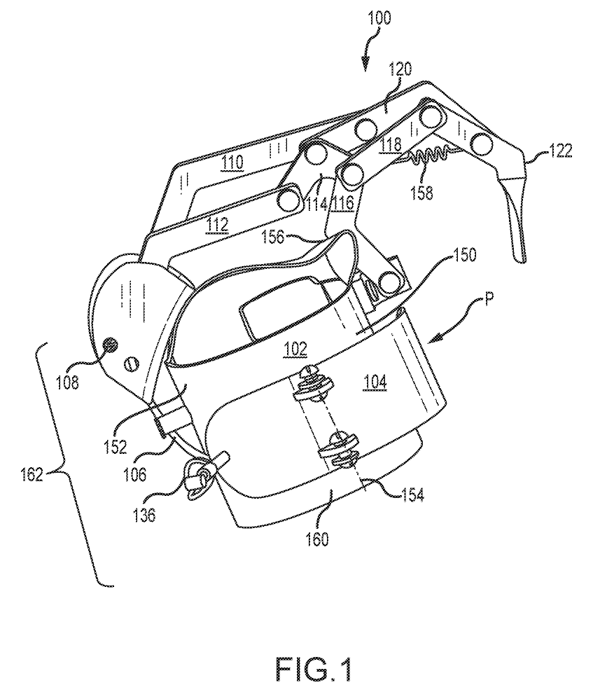

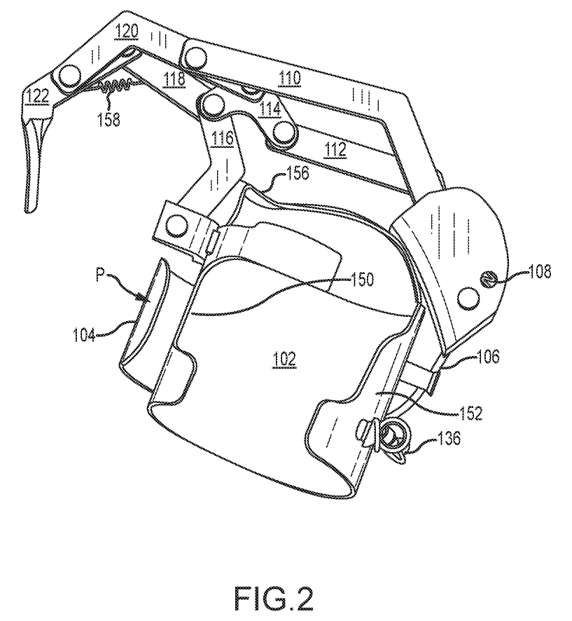

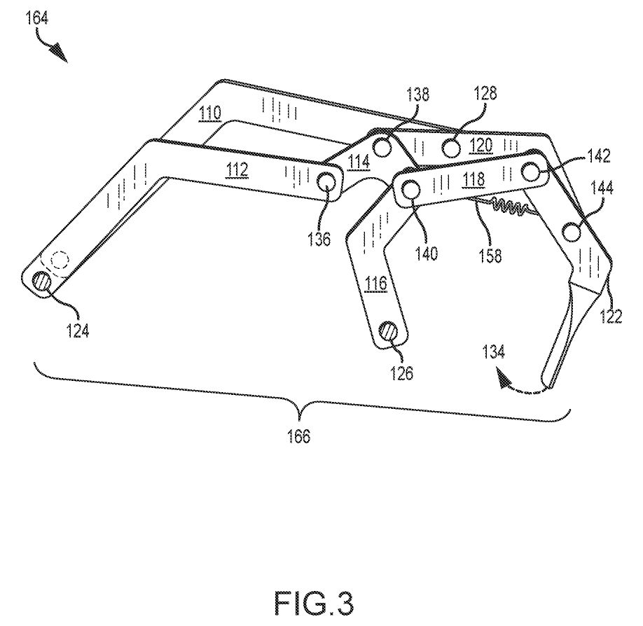

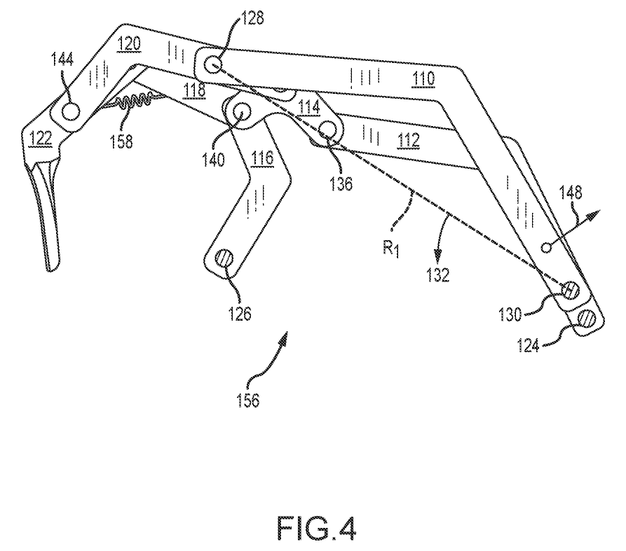

Turning now to FIG. 1 and FIG. 2, a partial hand prosthesis 100 is now described in detail. Generally, the partial hand prosthesis 100 is a prosthesis that is configured to provide functionality for one or more missing digits. The prosthesis 100 shown is configured to provide functionality for a missing third digit; however, those skilled in the art will recognize that the prosthesis 100 may be configured for a user missing one or more of the first through fifth digits. The prosthesis 100 has a palm socket 102, a lever 104, a linkage system 164 (see e.g. FIG. 3 and FIG. 4), and a spring-cable system (see cable 106, spring 108, and spring 136).

The palm socket 102 is generally shaped and configured to engage the contours of a user’s hand substantially in a manner known to those of skill in the art. The palm socket 102 has a palmar portion 150 configured to engage a palmar portion of the user’s hand and a dorsal portion 152 configured to engage a dorsal portion of the user’s hand. The palm socket 102 may have a spacer 156, which may be an adjustable, rigid, or flexible spacer 156 to provide protection adjacent the area where the digit is missing. That is, the spacer 156 may assist in preventing the bone structure of the user’s hand from collapsing as a result of an amputation, such as may occur when the entirety of all three phalanges are removed, or a digit is entirely missing as a result of a birth defect. In some embodiments, the spacer 156 may be rigid to provide structural rigidity to the palm socket 102. In some embodiments, the spacer 156 may be shaped and configured to protect an amputation site.

Continuing with FIG. 1 and FIG. 2, the lever 104 may be pivotally coupled to the palm socket 102, and may be configured to rotate from a first position to a second position in response to a palmar-facing pressure P. That is, for example, a user may cause the lever 104 to rotate by pressing his or her palm against an object, such as a bicycle handlebar.

In some embodiments, the partial hand prosthesis 100 is configured to grasp a bicycle handlebar when the lever 104 is activated, and to automatically release the bicycle handlebar when the lever 104 is released. In some embodiments, the prosthesis 100 is configured to grasp a vehicle steering wheel when the lever 104 is activated, and to automatically release the vehicle steering wheel when the lever 104 is released. Those skilled in the art will recognize that these embodiments may require a fast-acting spring-cable system 162 and/or fast-acting linkage system 164 and/or fast-acting lever 104.

The linkage system 164 may be shaped and configured to simulate a finger-like motion in response to activation of the spring-cable system 162, and the spring-cable system 162 may be configured to respond to rotation of the lever 104. For example, rotation of the lever 104 relative to the palm socket 102 may cause a cable 106 to activate the linkage system 164. The spring-cable system 162 may include a first spring 136 configured to bias the lever 104 in an open configuration (shown) and a second spring 108 configured to bias the linkage system 164 in an open configuration (shown). When the user presses his or her hand against an object, a pressure P may overcome the bias of the springs 108, 136 to activate the linkage system 164 and cause the linkage system 164 to move from the open configuration to a closed configuration wherein the linkage system 164 may grasp or wrap around an object, such as a bicycle handlebar.

Put another way, the linkage system 164 may be configured to move from the open configuration to a closed configuration in response to activation of the spring-cable system 162 in a manner known to those skilled in the art, and the closed configuration of the linkage system 164 may be configured to simulate a grasping position of a finger.

As is most clearly seen in FIG. 2, at least a portion of the lever 104 may be positioned adjacent the palmar portion 150 of the palm socket 102.

Turning now to FIG. 3 and FIG. 4, the linkage system 164 is discussed in further detail. The linkage system 164 may be configured to substantially simulate the motion and grasping ability of a user’s finger. The linkage system 164 may have a driver link 110 and a follower linkage system 166. The driver link 110 may be configured to respond to activation of the lever 104, and the follower linkage system 166 may be responsive to the driver link 110. The driver link 110 may have a proximal fixed pivot point 130 (see FIG. 4) and a rotatable distal end pivotally coupled to the follower linkage system 166. The driver link 110 may be coupled to the follower linkage system 166 at pivot link 128.

Continuing with FIG. 3 and FIG. 4, the follower linkage system 166 may have six linkage bars, such as first linkage bar 112, second linkage bar 116, third linkage bar 120, fourth linkage bar 114, fifth linkage bar 122, and sixth linkage bar 118. The first linkage bar 112 may have a proximal fixed pivot point 124 dorsal of the palm socket 102. The second linkage bar 116 may have a proximal fixed pivot point 126 palmar of the palm socket 102. The third linkage bar 120 may be pivotally coupled to a distal portion of the driver link 110, such as at pivot point 128. A proximal portion of the third linkage bar 120 may further be pivotally coupled a fourth linkage bar 114, such as at pivot point 138, seen most clearly in FIG. 3.

A distal portion of the third linkage bar 120 may be further pivotally coupled to a fifth linkage bar 122, such as at pivot point 144. A distal portion of the second linkage bar 116 may be pivotally coupled to a proximal portion of a sixth linkage bar 118 in the follower linkage system and a distal portion of the fourth linkage bar 114, such as at pivot point 140. A distal portion of the sixth linkage bar 118 may be further pivotally coupled to a proximal portion of the fifth linkage bar 122, such as at pivot point 142.

A medial portion of the fifth linkage bar 122 may be pivotally coupled to a distal portion of the third linkage bar 120, such as at pivot point 144.

In some embodiments, each of the first, second, third, and fourth linkage bars 112, 116, 120, 114 comprises a bend, each bend having an angle of greater than ninety degrees and less than one hundred eighty degrees, between the proximal end and the distal end, respectively.

In some embodiments, a medial portion of the fourth linkage bar 114 is pivotally coupled to the proximal portion of the third linkage bar 120, such as at pivot point 138, a proximal portion of the fourth linkage bar 114 is pivotally coupled to a distal portion of the first linkage bar 112, such as at pivot point 136, and a distal portion of the fourth linkage bar 114 is pivotally coupled to the distal portion of the second linkage bar 116 and the proximal portion of the sixth linkage bar 118, such as at pivot point 140.

In some embodiments, the fifth linkage bar 122 has a distal portion shaped and configured to simulate a distal portion or distal phalange of a human digit.

In some embodiments, the second, sixth, and fifth linkage bars 116, 118, 122 are shaped, positioned, and configured to rotate about an object in a grasping motion in response to an activation of the lever. That is, when the driver link 110 rotates about the pivot 130, pivot point 128 is forced to rotate at a distance of R1 from the pivot 130 to drive the follower linkage system 164; this will force the first linkage bar 112 to rotate about pivot point 124 and cause pivot point 136 to rotate at a distance of R2 from point 124 and the second linkage bar 116 to rotate as well. In response, the sixth linkage bar 118 and the fifth linkage bar 122 will move both linearly and pivotally, to grasp an object positioned between the second linkage bar 116 and the fifth linkage bar 122.

With continued reference to FIGS. 1-4, a limiter 158 may be provided. The limiter 158 may be configured to limit a distance between a proximal portion (e.g. 140) of the sixth linkage bar 118 and a distal portion of the fifth linkage bar 122, whereby the fifth and sixth linkage bars maintain an angle of less than one 180 degrees relative to each other. This limiter 158 may be a spring bias or an aesthetic feature such as a skin feature (not shown) in the prosthetic 100. That is, those skilled in the art will recognize that a skin feature may provide the limiting means required to prevent linkage bars 118 and 122 from buckling in the wrong direction in response to activation of the lever 104.

With reference to FIG. 4, in some embodiments, the proximal fixed pivot point 124 of the first linkage bar 112 in the follower linkage system 166 may be positioned proximal of the proximal fixed pivot point 130 of the driver link 110.

Returning now to FIGS. 1 and 2, the lever 104 may be pivotally coupled to the palm socket 102 adjacent to, and dorsal of, a fifth digit dorsal section 160 of the palm socket 102, such as at pivot point 154. The term “fifth digit dorsal section” should be understood to reference the most medial portion of the hand in the even the user does not have a fifth digit ray.

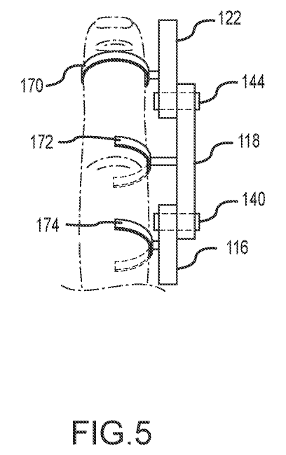

Although the prosthesis 100 has generally been described herein with terms that may imply use for users with a missing digit, it should be understood that the prosthesis 100 may be suitable and/or configured for use by a user who is not missing any digits, but instead requires a strengthened grip. For example, and with brief reference to FIG. 5, which show a portion of the linkage system 164, one or more finger sockets 170, 172, 174 may be affixed to one or more linkage bars, such as one or more of linkage bars 116, 118, 122 in the linkage system 164. One or more of the finger sockets 170, 172, 174 may be in the shape of a ring, such as shown with respect to finger socket 170. One ore more of the finger sockets 170, 172, 174 may be a u-socket, such as shown with respect to finger socket 174. It should be understood that any suitable shape may be used to provide comfort to the user. The finger socket(s) 170, 172, 174 may engage a user’s digit, whereby a grasping motion of the linkage system 164 effectuates a strengthened grasping motion of the user’s digit. In some embodiments, at least one of the linkage bars has a finger socket affixed thereto, the finger socket shaped and configured to engage a digit of the user adjacent the linkage system 164, whereby movement of the linkage system 164 effectuates movement of the digit. Those skilled in the art will recognize that, although FIG. 5 illustrates three finger sockets (possibly corresponding to three phalanges), only one or two finger sockets may be provided or used, depending on the user’s needs and preferences.

As demonstrated, the prosthesis 100 described herein provides a fast-acting mechanical means to cause a prosthetic digit to close about an object in response to a lever activation, and to automatically release the object when the lever is no longer activated, thereby providing an intuitive grasping and release action for the user that is not dependent on hydraulics or activation by the opposing hand and improving daily activities.

Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, the disclosure of a “attachment mechanism” should be understood to encompass disclosure of the act of “attaching” —whether explicitly discussed or not—and, conversely, were there only disclosure of the act of “attaching”, such a disclosure should be understood to encompass disclosure of a “attaching mechanism”. Such changes and alternative terms are to be understood to be explicitly included in the description.

Moreover, the claims shall be construed such that a claim that recites “at least one of A, B, or C” shall read on a device that requires “A” only. The claim shall also read on a device that requires “B” only. The claim shall also read on a device that requires “C” only. The claim shall also read on a device that requires “A+B”. The claim shall also read on a device that requires “A+B+C”, and so forth.

Those skilled in the art can readily recognize that numerous variations and substitutions may be made in the invention, its use and its configuration to achieve substantially the same results as achieved by the embodiments described herein.

Accordingly, there is no intention to limit the invention to the disclosed exemplary forms. Many variations, modifications and alternative constructions fall within the scope and spirit of the invention as expressed in the claims.

1. A partial hand prosthesis, comprising:
a palm socket shaped and configured to engage the contours of a user’s hand, the palm socket having a palmar portion configured to engage a palmar portion of the user’s hand and a dorsal portion configured to engage a dorsal portion of the user’s hand;
a lever pivotally coupled to the palm socket, the lever configured to rotate from a first position to a second position in response to a palmar-facing pressure;
a linkage system; and
a spring-cable system; wherein
the linkage system is shaped and configured to simulate a finger-like motion in response to activation of the spring-cable system; and wherein
the spring-cable system is configured to respond to rotation of the lever.

2. The partial hand prosthesis of claim 1, wherein:
at least a portion of the lever is positioned adjacent the palmar portion of the palm socket.

3. The partial hand prosthesis of claim 1, wherein:
the spring-cable system comprises a first resilient member shaped, configured, and positioned to bias the lever toward the first position; and
a second resilient member shaped, configured, and positioned to bias the linkage system in an open configuration.

4. The partial hand prosthesis of claim 3, wherein:
the linkage system is configured to move from the open configuration to a closed configuration in response to activation of the spring-cable system.

5. The partial hand prosthesis of claim 4, wherein:
the closed configuration of the linkage system is configured to simulate a grasping position of a finger.

6. The partial hand prosthesis of claim 1, wherein:
the palm socket comprises a spacer portion shaped and configured to at least one of:
protect an amputation area of the user; or
provide structural rigidity for the palm socket.

7. The partial hand prosthesis of claim 1, wherein:
the linkage system comprises a driver link and a follower linkage system;
the driver link has a proximal fixed pivot point and a rotatable distal end pivotally coupled to the follower linkage system; and wherein
the follower linkage system comprises six linkage bars.

8. The partial hand prosthesis of claim 7, wherein:
the six linkage bars in the follower linkage system comprise a first linkage bar having a proximal fixed pivot point dorsal of the palm socket and a second linkage bar having a proximal fixed pivot point palmar of the palm socket.

9. The partial hand prosthesis of claim 7, wherein:
the six linkage bars in the follower linkage system comprise a third linkage bar pivotally coupled to a distal portion of the driver link.

10. The partial hand prosthesis of claim 9, wherein:
a proximal portion of the third linkage bar is further pivotally coupled a fourth linkage bar in the follower linkage system; and
a distal portion of the third linkage bar is further pivotally coupled to a fifth linkage bar in the follower linkage system.

11. The partial hand prosthesis of claim 10, wherein:
a distal portion of the second linkage bar is pivotally coupled to a proximal portion of a sixth linkage bar in the follower linkage system and a distal portion of the fourth linkage bar; and wherein
a distal portion of the sixth linkage bar is further pivotally coupled to a proximal portion of the fifth linkage bar.

12. The partial hand prosthesis of claim 11, wherein:
a medial portion of the fifth linkage bar is pivotally coupled to a distal portion of the third linkage bar.

13. The partial hand prosthesis of claim 11, wherein:
each of the first, second, third, and fourth linkage bars comprises a bend, each bend having an angle of greater than ninety degrees and less than one hundred eighty degrees.

14. The partial hand prosthesis of claim 13, wherein:
a medial portion of the fourth linkage bar is pivotally coupled to the proximal portion of the third linkage bar;
a proximal portion of the fourth linkage bar is pivotally coupled to a distal portion of the first linkage bar; and
a distal portion of the fourth linkage bar is pivotally coupled to the distal portion of the second linkage bar and the proximal portion of the sixth linkage bar.

15. The partial hand prosthesis of claim 11, wherein:
the fifth linkage bar comprises a distal portion shaped and configured to simulate a distal portion of a human digit.

16. The partial hand prosthesis of claim 11, wherein:
the second, sixth, and fifth linkage bars are shaped, positioned, and configured to rotate about an object in a grasping motion in response to an activation of the lever.

17. The partial hand prosthesis of claim 16, further comprising:
a limiter configured to limit a distance between a proximal portion of the sixth linkage bar and a distal portion of the fifth linkage bar, whereby the fifth and sixth linkage bars maintain an angle of less than one 180 degrees relative to each other.

18. The partial hand prosthesis of claim 7, wherein:
at least one of the linkage bars has a finger socket affixed thereto, the finger socket shaped and configured to engage a digit adjacent the linkage system, whereby movement of the linkage system effectuates movement of the digit.

19. The partial hand prosthesis of claim 8, wherein:
the proximal fixed pivot point of the first linkage bar in the follower linkage system is positioned proximal of the proximal fixed pivot point of the driver link.

20. The partial hand prosthesis of claim 1, wherein:
the lever is pivotally coupled to the palm socket adjacent to, and dorsal of, a fifth digit dorsal section of the palm socket.

21. The partial hand prosthesis of claim 1, wherein:
the partial hand prosthesis is configured to at least one of:
grasp a bicycle handlebar when the lever is activated, and to automatically release the bicycle handlebar when the lever is released; or
grasp a vehicle steering wheel when the lever is activated, and to automatically release the vehicle steering wheel when the lever is released.
